SEQUENCE LISTING (1 sequence)

SEQ ID NO: 1 - protein, 291 aa, Artificial sequence
  Protein translated from sythetic DNA
MTKEYQDLQHLDNEESDHHQLRKGPPPPQPLLQRLCSGPRLLLLSLGLSLLLLVVVCVIGSQNSQLQEELRGLRETFSNFTASTEAQVKGLSTQGGNVGRKMKSLESQLEKQQKDLSEDHSSLLLHVKQFVSDLRSLSCQMAALQGNGSERTCCPVNWVEHERSCYWFSRSGKAWADADNYCRLEDAHLVVVTSWEEQKFVQHHIGPVNTWMGLHDQNGPWKWVDGTDYETGFKNWRPEQPDDWYGHGLGGGEDCAHFTDDGRWNDDVCQRPYRWVCETELDKASQEPPLL

(19) United States
(12) Patent Application Publication
(10) Pub. No.: US 2020/0319174 A1
(43) Pub. Date: Oct. 8, 2020

(54) Method for Detecting Human Soluble Asialoglycoprotein Receptor

(71) Applicant: Jiangnan University, Wuxi (CN)

(72) Inventors: Jing HU, Wuxi (CN); Jian YIN, Wuxi (CN); Qimin SHI, Wuxi (CN)

(21) Appl. No.: 16/906,106

(22) Filed: Jun. 19, 2020

Related U.S. Application Data
(63) Continuation of application No. PCT/CN2019/112461, filed on Oct. 22, 2019.

(30) Foreign Application Priority Data
Apr. 17, 2019 (CN) 2019103082415

(51) Int. Cl. G01N 33/543; G01N 33/68; C07K 14/765

(52) U.S. Cl. CPC G01N 33/54306; C07K 14/765; G01N 33/68

(57) ABSTRACT

The disclosure discloses a method for detecting a human soluble asialoglycoprotein receptor, and belongs to the field of immunological detection. The disclosure provides an economical, rapid, accurate and highly practical ELISA method for detecting a human sASGPR. Based on the specific recognition between a specific ligand of ASGPR and ASGPR, the method selects galactosylated human serum albumin (GSA) as the specific ligand of ASGPR. GSA is prepared from human serum albumin, and has the advantages of cheap price, easy operation, easy storage and the like, and the limit of detection of the method is suitable for the detection of sASGPR in human serum samples. The method can be carried out in ordinary laboratories without using special, large-scale instruments and equipment. The method has the advantages of high specificity, good stability, simple and convenient operation, low cost and the like, and provides a certain reference value for clinical liver function evaluation.

### TECHNICAL FIELD

The disclosure herein relates to a method for detecting a human soluble asialoglycoprotein receptor, and belongs to the field of immunological detection.

### BACKGROUND

Liver disease has been an important high-morbidity disease in the world for decades. Liver injury such as cirrhosis, hepatitis and fatty liver may be eventually developed into liver cancer. Liver injury seriously threatens the health and life of people in China and even the world. At present, the main detection method for liver injury includes histopathological diagnosis, serological diagnosis and imaging diagnosis. The main clinical serum marker for serological diagnosis includes alanine aminotransferase (ALT), aspartate aminotransferase (AST), alkaline phosphatase (ALP) and serum bilirubin (BIL), etc., but these indexes have low specificity. Except that the liver injury will affect these indexes, some other diseases will also cause abnormal changes in these indexes. Therefore, it is necessary to discover a novel serum marker of liver injury, which can specifically indicate abnormal liver function.

A soluble asialoglycoprotein receptor (sASGPR) is a soluble protein specifically expressed by the liver. Soluble galactose/N-acetylgalactosamine sugar ligands will appear in the blood due to normal tissue metabolism, injury, diet, disease and other factors to competitively bind lectins between cells and tissues. sASGPR in the blood is mainly involved in the removal of these harmful sugar ligands, thereby maintaining the stability of galactose levels in the body. Studies have shown that the content of sASGPR in human blood is maintained at a certain constant level in healthy people, and when liver injury occurs, it will cause changes in the content of sASGPR. Therefore, as a novel potential liver function marker, sASGPR is attracting the attention of researchers. If the relation between sASGPR and liver injury can be established, it will provide great help for clinical detection of liver function.

At present, there are few commercially available kits for detecting the human sASGPR, and the existing ELISA kits for detecting the human ASGPR are all double-antibody sandwich kits. The double-antibody sandwich kit requires the use of two different antibodies against different sites of ASGPR. The antibody preparation process is time-consuming and labor-consuming, and the preparation cost is relatively high, resulting in the kit being relatively expensive and the user detection cost being increased; in addition, the limit of detection of some existing ELISA kits is also not suitable for the detection of sASGPR in human serum, so that the detection operation on serum samples is inconvenient and the results are not accurate enough at the same time.

### SUMMARY

The disclosure provides an economical, rapid, accurate and highly practical ELISA method for detecting a human sASGPR. The ELISA method is an indirect ELISA method established based on the specific recognition between a specific ligand of ASGPR and ASGPR. Compared with the common double-antibody sandwich ELISA method, the method selects galactosylated human serum albumin (GSA) as the specific ligand of ASGPR. GSA is prepared from human serum albumin, and has the advantages of cheap price, easy operation, easy storage and the like, and the limit of detection of the method is suitable for the detection of sASGPR in human serum samples, and the practicability is high. The method can be carried out in ordinary laboratories without using special, large-scale instruments and equipment.

A first object of the disclosure is to provide a method for detecting a human soluble asialoglycoprotein receptor. An ELISA method is used for detecting by using galactosylated human serum albumin (GSA) as a specific ligand of ASGPR.

In one embodiment of the disclosure, a method for preparing the galactosylated human serum albumin (GSA) is as follows:

20 mg of human serum albumin is added to a clean reaction flask, then 20 mL of a 0.5 M MES biological buffer (pH 5.25) is added to fully dissolve the human serum albumin, then 108.6 mg of galactosamine and 62 mg of EDC are sequentially added to fully dissolve, and after the sample addition is fully completed, the reaction flask is put in a 37° C. oil bath pan to react for 16 h; after the reaction is finished, 3.4 mL of 1 M CH₃COOH solution (pH 4.5) is added to the reaction flask to stop the reaction; after the reaction is stopped, a reaction solution is transferred to an Amicon® MLtra centrifugal filter for centrifugation at 5000×g for 20 min, a filtrate in the lower centrifuge tube is discarded, a 10 mM CH₃COOH solution (pH 7) is added to the upper filter to restore to the original sample volume, and centrifuging is performed again at 5000×g, and ultrafiltration is repeated many times to remove unreacted galactosamine and reduce the concentration of sodium acetate; a GSA solution is finally obtained after performing ultrafiltration many times.

In one embodiment of the disclosure, the method specifically includes: coating an ELISA plate with GSA diluted with a CBS buffered solution at a temperature of 4° C. for 12 to 24 h, and then washing with a washing buffer; adding a blocking buffer to the coated ELISA plate to block at a temperature of 35 to 37° C. for 1.5 to 2.5 h; discarding the blocking buffer, and washing with a washing buffer; adding a sample to the ELISA plate, incubating at a temperature of 35 to 37° C. for 2 to 2.5 h, then washing with a washing buffer, and patting dry; adding an ASGPR1 primary antibody and incubating at a temperature of 35 to 37° C. for 2 to 2.5 h, and then washing with a washing buffer; adding an HRP-labeled goat anti-mouse enzyme-labeled secondary antibody, incubating at a temperature of 35 to 37° C. for 1 to 2 h, and washing with a washing buffer; developing with a TMB development solution, incubating for 10 to 20 min in the dark, and then immediately stopping the development with H₂SO₄; and determining the absorbance at 450 nm.

In one embodiment of the disclosure, the concentration of the CBS buffered solution is 0.05 M.

In one embodiment of the disclosure, the coating concentration of the GSA is 15 to 25 μg/m L.

In one embodiment of the disclosure, the blocking buffer is phosphate buffered solution Tween (PBST) containing 1% skim milk.

In one embodiment of the disclosure, the amino acid sequence of the ASGPR primary antibody is shown in SEQ ID NO.1.

In one embodiment of the disclosure, the ASGPR primary antibody is added after being diluted 50 times; the ASGPR primary antibody is purchased from Santa Cruz Biotechnology, and the article number is sc-166633.

In one embodiment of the disclosure, the enzyme-labeled secondary antibody is added after being diluted 2000 times; the enzyme-labeled secondary antibody is purchased from Kangwei Century Biotechnology Co., Ltd., and the article number is CW0102.

In one embodiment of the invention, the development is stopped with H₂SO₄ having a concentration of 2 M.

In one embodiment of the disclosure, the method includes the following specific steps:

(1) Coating: diluting GSA with a 0.05 M CBS buffered solution to 20 μg/mL to coat a 96-well ELISA plate with 100 μL per well at a temperature of 4° C. for 24 h, then washing with PBS containing 0.1% tween-20 three times, 3 min each time, and patting dry;

(2) Blocking: adding 1% skim milk to the coated ELISA well plate with 300 μL per well, sealing the plate with a microplate sealer, and blocking at a temperature of 37° C. for 2 h; discarding the blocking buffer, washing with PBS containing 0.1% tween-20 three times, 3 min each time, and patting dry;

(3) Sample adding: adding a sample to be detected to a 96-well ELISA plate, the addition volume of the sample per well being 100 μL; after adding the sample, sealing the plate with a microplate sealer, and incubating at a temperature of 37° C. for 2 h.

(4) Primary antibody incubation: incubating a mouse-derived primary antibody of ASGPR1/2 by diluting with a blocking buffer according to a ratio of 1:50, with 100 μL per well, incubating at a temperature of 37° C. for 2 h, then washing with PBS containing 0.1% tween-20 four times, 3 min each time, and patting dry;

(5) Secondary antibody incubation: incubating an HRP-labeled goat anti-mouse enzyme-labeled secondary antibody by diluting with a blocking buffer according to a ratio of 1:2000, with 100 μL per well, incubating at a temperature of 37° C. for 1 h, then washing with PBS containing 0.1% tween-20 four times, 3 min each time, and patting dry;

(6) TMB development: developing with a TMB development solution (Biyuntian Biotechnology Co., Ltd., P0209), with 200 μL per well, incubating for 15 min in the dark, and then immediately stopping the development with 2 M H₂SO₄, with 50 μL per well; after stopping the development, determining the absorbance of each well at 450 nm with an ELISA as soon as possible.

In one embodiment of the disclosure, the ELISA plate is prepared by using a 96-well plate.

A second object of the disclosure is to provide a detection kit, including a GSA-coated ELISA plate, a blocking buffer, an ASGPR standard product, an ASGPR primary antibody, a goat anti-mouse enzyme-labeled secondary antibody, a stop buffer and a washing buffer.

In one embodiment of the disclosure, the GSA-coated ELISA plate is prepared by the following method including: diluting GSA to 20 μg/mL with a coating solution, coating a 96-well ELISA plate with 100 μL per well at a temperature of 4° C. for 24 h, then washing with a washing buffer (PBS containing 0.1% tween-20) three times, 3 min each time, and patting dry.

In one embodiment of the disclosure, the coating solution is a CBS buffered solution, which is prepared by the method including: weighing 1.59 g of Na₂CO₃ and 2.93 g of NaHCO₃ and dissolving in 800 mL of ddH₂O, adjusting the pH of the solution to 9.6 with NaOH, and finally, accurately metering the volume to 1 L with ddH₂O, and storing at a temperature of 4° C.

In one embodiment of the disclosure, the washing buffer includes a first washing buffer and a second washing buffer; the first washing buffer is a 0.01 M PBS buffered solution, which is prepared by the method including: weighing 2.9 g of Na₂HPO₄.12H₂O, 0.2 g of KH₂PO₄, 8 g of NaCl, and 0.2 g of KCl and dissolving in 800 mL of ddH₂O, adjusting the pH of the solution to 7.4 with HCl, and finally, accurately the volume to 1 L with ddH₂O, and storing at a temperature of 4° C.; the second washing buffer is a PBST buffered solution, which is prepared by the method including: adding 1 mL of Tween 20 to 1 L of the prepared 0.01 M PBS buffered solution, mixing well, and storing at a temperature of 4° C.

In one embodiment of the disclosure, the blocking buffer is prepared by the method including: weighing 1 g of skim milk and dissolving in 100 mL of a PBST buffered solution, after fully dissolving, filtering with a 0.22 μm sterile filter membrane and then storing at a temperature of 20° C.

In one embodiment of the disclosure, a method for preparing an ASGPR standard diluent includes: taking 1 mL of a 1 M NaCl solution prepared in advance, 100 μL of a 1 M Tris-HCl solution (pH 7.4), and 100 μL of a 1 M CaCl₂ solution and placing in a beaker, and accurately weighing 0.1 g of BSA and adding to the beaker, accurately metering the volume to 10 mL with ddH₂O, after fully dissolving, filtering with a 0.22 μm sterile filter membrane and then storing at a temperature of 20° C.

In one embodiment of the disclosure, the ASGPR primary antibody is purchased from Santa Cruz Biotechnology, and the article number is sc-166633; the enzyme-labeled secondary antibody is purchased from Kangwei Century Biotechnology Co., Ltd., and the article number is CW0102; the TMB development solution is purchased from Biyuntian Biotechnology, and the article number is P0209.

In one embodiment of the disclosure, the stop buffer is 2 M H₂SO₄, which is prepared by the method including: adding 89.15 mL of ddH₂O to a beaker, and then adding dropwise 10.85 mL of concentrated sulfuric acid (98%) while slowly stirring.

The disclosure also claims to protect the application of the kit in detecting a liver function index.

Beneficial effects: by selecting galactosylated human serum albumin (GSA) as the specific ligand of ASGPR instead of the expensive antibody-coated ELISA plate, and optimizing the conditions of ELISA detection, a detection method suitable for sASGPR in human serum samples is established, and the limit of detection can reach 4 μg/mL; the average values of coefficients of variation of inter-batch and intra-batch repeat tests are all less than 7%, and the repeatability is better; and the linearly dependent coefficients r of fitted straight lines obtained by repeat tests are all greater than 0.990, and the method satisfies the expected set linear range of 4 to 100 μg/mL; after being stably stored at the temperature of 37° C. for 10 days, the ELISA plate well-coated by applying the method of the disclosure can still achieve good repeatability and linear range, the limit of detection complies with the standard, and the application prospects are good.

### BRIEF DESCRIPTION OF FIGURES

FIG. 1 is a schematic diagram of an ELISA method for detecting a human sASGPR.

FIG. 2 shows the results of SDS-PAGE gel electrophoresis (A) and Western Blot (B) of ASGPR standard products after purification.

FIG. 3 is a schematic diagram of an ASGPR standard curve.

FIG. 4 is a linear fit chart of determined concentration and theoretical concentration.

FIG. 5 shows the detection of an sASGPR in clinical serum samples.

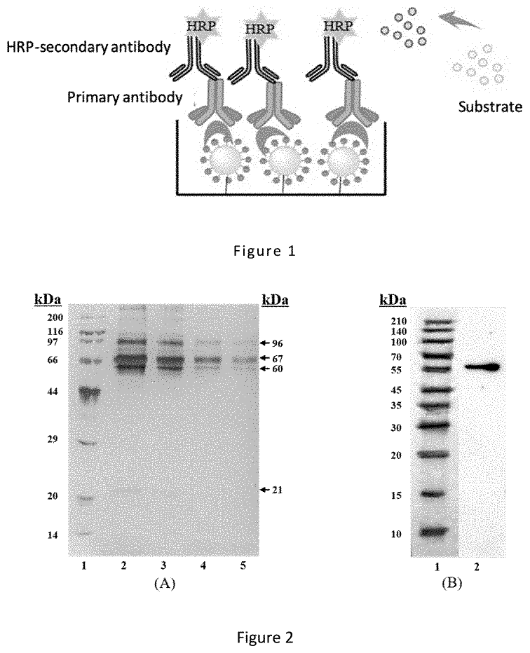

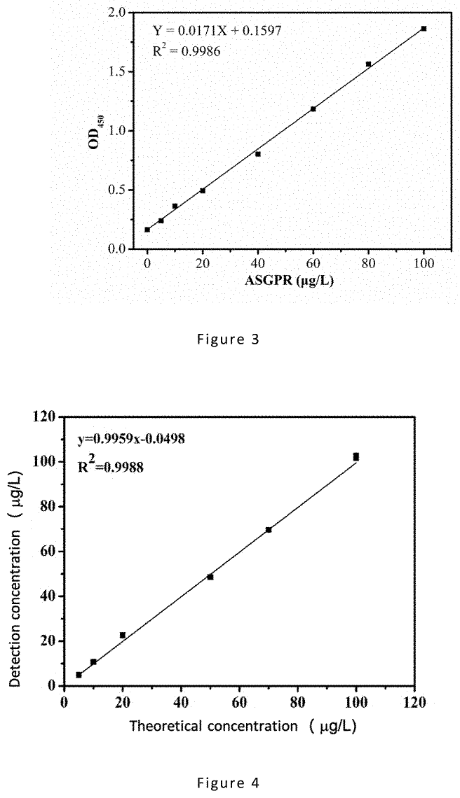

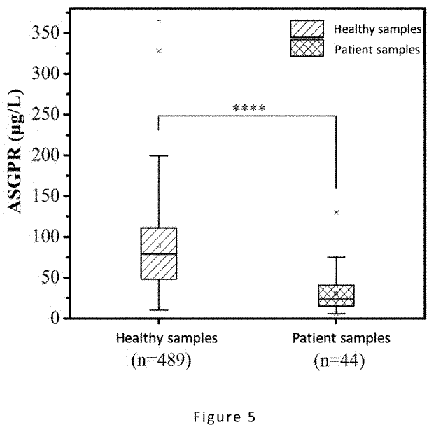

### DETAILED DESCRIPTION

### Example 1 Preparation of Ligand GSA

20 mg of human serum albumin was added to a clean reaction flask, then 20 mL of 0.5 M MES biological buffer (pH 5.25) was added to fully dissolve the human serum albumin, then 108.6 mg of galactosamine and 62 mg of EDC were sequentially added to fully dissolve, and after the sample addition was fully completed, the reaction flask was put in a 37° C. oil bath pan to react for 16 h; after the reaction was finished, 3.4 mL of 1 M CH₃COOH solution (pH 4.5) was added to the reaction flask to stop the reaction; after the reaction was stopped, a reaction solution was transferred to an Amicon® MLtra centrifugal filter for centrifugation at 5000×g for 20 min, a filtrate in the lower centrifuge tube was discarded, a 10 mM CH₃COOH solution (pH 7) was added to the upper filter to restore to the original sample volume, and centrifuging was performed again at 5000×g, and ultrafiltration was repeated many times to remove unreacted galactosamine and reduce the concentration of sodium acetate; a GSA solution was finally obtained after performing ultrafiltration many times, subpackaged and put in a refrigerator at −80° C. for storing for later use. The protein concentration of GSA was detected by using a BCA kit (Takara, T9300A). Before determining, a solution A and a solution B of a BCA reagent were mixed according to the ratio of 100:1 and prepared into a working solution. The BSA standard solution was respectively diluted to 2000 μg/mL, 1500 μg/mL, 1000 μg/mL, 750 μg/mL, 500 μg/mL, 250 μg/mL, and 125 μg/mL, and then the diluted BSA standard solution and the GSA sample solution to be measured were sequentially added to a 96-well plate with 10 μL per well, two parallel samples were taken for each concentration, 200 μL of working solution was added to each well, the solutions were immediately mixed well, and then put in a 37° C. water bath to react for 30 min, and cooled to room temperature, and the absorbance at 562 nm was detected by an ELISA. The standard curve of a BSA standard solution was drawn, and the protein concentration of GSA protein to be measured was calculated according to the standard curve.

### Example 2 Purification of ASGPR Standard Product

150 mL of HepG2 cell supernatant collected in advance was taken and centrifuged for 5 min (3500×g) by a centrifuge, and the centrifuged supernatant was taken and added to a 50 mL Amicon® MLtra centrifugal filter (3500×g) for concentration, and finally concentrated to a final volume of 8 mL. 8 mL of lysis buffer was added to the concentrated HepG2 cell supernatant and mixed well, and was lysed for 2 to 3 h at a temperature of 4° C. After lysis was completed, centrifuging was performed at 20,000×g for 30 min, the supernatant was taken into a new centrifuge tube, and 800 μL of 1 M CaCl₂ was added to incubate on ice for 30 min. Centrifuging was performed again at 20000×g for 30 min, and a precipitate was discarded. A lactose agarose bead column was equilibrated with 50 mL of washing buffer I in advance, and lactose agarose beads were fully transferred to the centrifuge tube and combined with the centrifuged cell supernatant at a temperature of 4° C. overnight. The combined lactose agarose beads were fully transferred to the column, and the column was sequentially washed with 10 mL of washing buffer I and 5 mL of washing buffer II. The lactose agarose bead column was eluted with 14.4 mL of elution buffer, and 1.6 mL of 1 M Tris-HCl (pH 7.8) buffered solution was added to the collected eluent. The concentration of purified sASGPR was detected by a BCA method, and specificity was verified by Western blot.

### Example 3 Selection of Primary Antibodies

Two ASGPR1 mouse-derived primary antibodies, namely, ASGPR1/2 (E-1) (Santa Cruz Biotechnology, sc-166633) and ASGPR1 (A-5) (Santa Cruz Biotechnology, sc-393849), were purchased, hereinafter referred to as a mouse anti-human ASGPR-1 monoclonal antibody and a mouse anti-human ASGPR-2 monoclonal antibody. Two 96-well ELISA plates were coated with 10 μg/mL GSA, 100 μL of GSA was added to each well, coating was performed at a temperature of 4° C. for 24 h, after coating was finished, washing was performed with PBST 5 times, 3 min each time and patting dry was performed. 300 μL of 1% BSA was added to each well to block the whole well, blocking was performed overnight at a temperature of 4° C., after blocking was finished, washing was performed with PBST 3 times, 3 min each time and patting dry was performed. An ASGPR diluent was set as a negative control well and a quality control product well respectively, and 0.15 μg/mL, 0.50 μg/mL, 2.00 μg/mL, and 8.00 μg/mL ASGPR standard products were respectively set as sample wells, the negative control and samples were sequentially added to the ELISA plate, 4 replicate wells were made in the sample, and the sample adding volume for each well is 100 μL. Incubation was performed for 1 h in a 37° C. incubator after sample addition. After the sample incubation was finished, washing was performed with PBST 3 times, 3 min each time and patting dry was performed. The mouse anti-human ASGPR-1 monoclonal antibody and the mouse anti-human ASGPR-2 monoclonal antibody diluted according to a ratio of 1:500 were respectively added to the two ELISA plates, the sample addition volume was 100 μL in each well, and incubation was performed in a 37° C. incubator for 1 h. After the primary antibody incubation was finished, washing was performed with PBST 4 times, 3 min each time and patting dry was performed. The goat anti-mouse IgG-HRP diluted according to a ratio of 1:2000 was added to the two plates, the sample adding volume for each well was 100 μL, and incubation was performed in a 37° C. incubator for 1 h. After the secondary antibody incubation was finished, washing was performed with PBST 4 times, 3 min each time and patting dry was performed. 200 μL of TMB development solution was added to each well to develop in a 37° C. incubator for 15 min in the dark. After the development was finished, 50 μL of 2M H₂SO₄ was quickly added to each well to stop the development and mixed well. The absorbance of each well at 450 nm was immediately measured with the ELISA and the results were analyzed.

TABLE 1
Detection results of different ASGPR primary antibodies

| | | |
|---|---|---|
| Primary | Negative | ASGPR concentration (μg/mL) |

| antibodies | control | 0.15 | 0.50 | 2.00 | 8.00 |
|---|---|---|---|---|---|
| ASGPR-1 | 0.263 | 0.301 | 0.380 | 1.112 | 2.204 |
| ASGPR-2 | 0.218 | 0.202 | 0.218 | 0.238 | 0.569 |

It can be seen from the data in the table that when using the mouse anti-human ASGPR-1 monoclonal antibody, the value of OD₄₅₀ will have a significant gradient increase with the increase of the concentration of ASGPR, so the mouse anti-human ASGPR-1 monoclonal antibody was selected as the detection primary antibody for the indirect ELISA method.

### Example 4 Selection of Coating Buffers

A phosphate buffered solution (0.01 M PBS), a Tris hydrochloride buffered solution (0.01 M TBS), a carbonate buffered solution (0.05 M CBS), and 0.9% NaCl were respectively used as coating buffers to be selected. 10 μg/mL GSA was coated with 0.01 M PBS, 0.01 M TBS, 0.05 M CBS, and 0.9% NaCl into a 96-well ELISA plate, the volume of GSA was 100 μL in each well, and coating was performed at a temperature of 4° C. for 24 h. After coating was finished, washing was performed with PBST 5 times, 3 min each time and patting dry was performed. 300 μL of 1% BSA was added to each well to block the whole well, blocking was performed overnight at a temperature of 4° C., after blocking was finished, washing was performed with PBST 3 times, 3 min each time and patting dry was performed. A negative control well and standard ASGPR1 (80 μg/mL) and standard ASGPR2 (40 μg/mL) sample wells were respectively set, the negative control and the samples were sequentially added to the ELISA plate, 4 replicate wells were made in the sample, and the sample adding volume for each well is 100 μL. Incubation was performed for 1 h in a 37° C. incubator after sample addition. After the sample incubation was finished, washing was performed with PBST 3 times, 3 min each time and patting dry was performed. The mouse anti-human ASGPR-1 monoclonal antibody diluted according to a ratio of 1:500 was respectively added to the two plates, the sample adding volume for each well was 100 μL, and incubation was performed in a 37° C. incubator for 1 h. After the primary antibody incubation was finished, washing was performed with PBST 4 times, 3 min each time and patting dry was performed. The goat anti-mouse IgG-HRP diluted according to a ratio of 1:2000 was added to the two plates, the sample adding volume for each well was 100 μL, and incubation was performed in a 37° C. incubator for 1 h. After the secondary antibody incubation was finished, washing was performed with PBST 4 times, 3 min each time and patting dry was performed. 200 μL of TMB development solution was added to each well to develop in a 37° C. incubator for 15 min in the dark. After the development was finished, 50 μL of 2 M H₂SO₄ was quickly added to each well to stop the development and mixed well. The absorbance of each well at 450 nm was immediately measured with the ELISA.

TABLE 2
Detection effects of different coating buffers

| | Samples | | | | |
|---|---|---|---|---|---|
| | | | Negative | P/N | |
| Coating buffers | ASGPR1 | ASGPR2 | control | P1/N | P2/N |
| 0.01M PBS | 1.682 | 0.958 | 0.234 | 7.19 | 4.09 |
| 0.01M TBS | 1.637 | 0.933 | 0.229 | 7.15 | 4.07 |
| 0.05M CBS | 1.882 | 1.042 | 0.202 | 9.32 | 5.16 |
| 0.9% NaCl | 1.598 | 0.932 | 0.212 | 7.54 | 4.40 |

It can be seen from the data in the table that when using 0.05 M CBS as the coating buffer, the value of positive sample OD₄₅₀/negative control OD₄₅₀ (P/N) is the highest.

### Example 5 Selection of Different Coating Concentrations

GSA was diluted with 0.05 M CBS to 5 μg/mL, 10 μg/mL, 15 μg/mL, 20 μg/mL, and 25 μg/mL, respectively, and coated on different ELISA plates, with 100 μL volume per well. The rest of the steps are the same as above, and the OD₄₅₀ values of the negative control well and the standard product ASGPR1 (80 μg/mL) and standard product ASGPR2 (40 μg/mL) sample wells were detected respectively. 4 replicate wells were set in each sample for detecting.

TABLE 3
Test results of different coating concentrations

| GSA coating | | Samples | | | | |
|---|---|---|---|---|---|---|
| concentrations | | | | Negative | P/N | |
| (μg/mL) | | ASGPR1 | ASGPR2 | control | P1/N | P2/N |
| 5 | μg/mL | 1.207 | 0.427 | 0.146 | 8.26 | 2.92 |
| 10 | μg/mL | 1.256 | 0.464 | 0.147 | 8.57 | 3.17 |
| 15 | μg/mL | 1.404 | 0.577 | 0.159 | 8.83 | 3.63 |
| 20 | μg/mL | 1.432 | 0.711 | 0.151 | 9.48 | 4.71 |
| 25 | μg/mL | 1.435 | 0.720 | 0.164 | 8.86 | 4.45 |

The results show that when coating 20 μg/mL GSA, the P/N value is the highest.

### Example 6 Selection of Coating Time

20 μg/mL GSA was coated onto the ELISA plate with 0.05 M CBS. The coating conditions were respectively set as follows: coating at a temperature of 37° C. for 1 h, coating at a temperature of 37° C. for 2 h, coating at a temperature of 4° C. for 12 h, and coating at a temperature of 4° C. for 24 h. The rest of the steps are the same as above. The OD₄₅₀ values of the negative control well and standard product ASGPR1 (80 μg/L) and standard product ASGPR2 (40 μg/L) sample wells were respectively detected, 4 replicate wells were set in each sample for detecting, and the optimal GSA coating time was determined according to the P/N value.

TABLE 4
Detection results of different coating time

| | Samples | | | | |
|---|---|---|---|---|---|
| | | | Negative | P/N | |
| Coating time | ASGPR1 | ASGPR2 | control | P1/N | P2/N |
| 1 h at 37° C. | 1.225 | 0.722 | 0.255 | 4.80 | 2.83 |
| 3 h at 37° C. | 1.375 | 0.776 | 0.209 | 6.57 | 3.71 |
| 12 h at 4° C. | 1.007 | 0.608 | 0.151 | 6.68 | 4.04 |
| 24 h at 4° C. | 1.106 | 0.679 | 0.149 | 7.43 | 4.56 |

The results show that when coating at a temperature of 4° C. for 24 h, the P/N value is the highest. Therefore, the coating time of 24 h at a temperature of 4° C. is selected as the coating time for the indirect ELISA method.

### Example 7 Selection of Blocking Buffers

20 μg/mL GSA was coated with 0.05 M CBS at a temperature of 4° C. for 24 h. Six different blocking buffers, i.e., 1% BSA, 5% BSA, 1% skim milk powder, 5% skim milk powder, 1% FBS, and 5% FBS were respectively used for blocking. 300 μL of blocking buffer was added to each well to block at a temperature of 37° C. for 1 h. The rest of the steps are the same as above, the OD₄₅₀ values of the negative control well and standard product ASGPR1 (80 μg/L) and standard product ASGPR2 (40 μg/L) sample wells were respectively detected, 4 replicate wells were set in each sample for detecting, and finally the optimal blocking buffer was determined according to the P/N value.

TABLE 5
Detection results of different blocking buffers

| | Samples | | | | |
|---|---|---|---|---|---|
| | | | Negative | P/N | |
| Blocking buffers | ASGPR1 | ASGPR2 | control | P1/N | P2/N |
| 1% BSA | 1.323 | 0.567 | 0.149 | 8.91 | 3.82 |
| 5% BSA | 1.403 | 0.613 | 0.155 | 9.08 | 3.97 |
| 1% skim milk | 1.512 | 0.726 | 0.134 | 11.32 | 5.43 |
| powder | | | | | |
| 5% skim milk | 1.429 | 0.577 | 0.134 | 10.70 | 4.32 |
| powder | | | | | |
| 1% FBS | 1.26 | 0.496 | 0.150 | 8.43 | 3.32 |
| 5% FBS | 1.217 | 0.492 | 0.151 | 8.06 | 3.26 |

When using 1% skim milk powder as the blocking buffer, the P/N value is the highest.

### Example 8 Selection of Blocking Time

20 μg/mL GSA was coated with 0.05 M CBS at a temperature of 4° C. for 24 h, and blocked with 1% skim milk powder. The blocking time was set as 1 h at a temperature of 37° C., 1.5 h at a temperature of 37° C., 2 h at a temperature of 37° C., 2.5 h at a temperature of 37° C., and 3 h at a temperature of 37° C. The rest of the steps are the same as above. The OD₄₅₀ values of the negative control well and standard product ASGPR1 (80 μg/L) and standard product ASGPR2 (40 μg/L) sample wells were respectively detected, 4 replicate wells were set in each sample for detecting, and finally the optimal blocking time was determined according to the P/N value.

TABLE 6
Detection effects of different blocking time

| | | Samples | | | | |
|---|---|---|---|---|---|---|
| | | | | Negative | P/N | |
| Blocking time | | ASGPR1 | ASGPR2 | control | P1/N | P2/N |
| 1 | h | 1.412 | 0.823 | 0.186 | 7.61 | 4.43 |
| 1.5 | h | 1.552 | 0.934 | 0.163 | 9.52 | 5.73 |
| 2 | h | 1.519 | 0.885 | 0.123 | 12.35 | 7.20 |
| 2.5 | h | 1.626 | 0.935 | 0.163 | 10.01 | 5.75 |
| 3 | h | 1.392 | 0.857 | 0.197 | 7.08 | 4.36 |

The results show that when the blocking time is 2 h, the P/N value is the highest.

### Example 9 Selection of Sample Incubation Time

Coating and blocking were performed by selecting the optimum conditions determined by the above experiments. The negative control and standard product ASGPR1 (80 μg/L) and standard product ASGPR2 (40 μg/L) samples were respectively detected, 4 replicate wells were set in each sample for detecting, the sample incubation time was respectively set as 1 h, 1.5 h, 2 h, 2.5 h, 3 h, and finally the optimal sample incubation time was determined according to the OD value and P/N value.

TABLE 7
Detection results of different sample incubation time

| | | Samples | | | | |
|---|---|---|---|---|---|---|
| Incubation | | | | Negative | P/N | |
| time | | ASGPR1 | ASGPR2 | control | P1/N | P2/N |
| 1 | h | 1.155 | 0.798 | 0.166 | 6.96 | 4.81 |
| 1.5 | h | 1.208 | 0.945 | 0.167 | 7.23 | 5.66 |
| 2 | h | 1.407 | 1.151 | 0.184 | 7.82 | 6.39 |
| 2.5 | h | 1.498 | 1.221 | 0.191 | 7.84 | 6.39 |

When the incubation time reaches 2 h, the P/N value reaches the highest, and when the incubation time increases again, although the OD value of the sample well will increase, the corresponding negative control value will also increase, and the P/N value remains stable.

### Example 10 Optimization of Extent of Dilution of Primary and Secondary Antibodies

Coating and blocking were performed by selecting the optimum conditions determined by the above experiments. The negative control well and ASGPR sample wells were respectively detected. 4 replicate wells were set in each sample for detecting. The primary antibodies were respectively diluted according to 1:30, 1:50, 1:100, 1:200, and 1:400, and the secondary antibodies were respectively diluted according to 1:1000, 1:2000, 1:4000, and 1:6000. Finally, the optimal extend of dilution of the secondary antibody was determined according to the P/N value.

TABLE 8
Detection effects of different extents of dilution of primary and secondary
antibodies

| | Extent of dilution of primary antibody | | | | | | | | | |
|---|---|---|---|---|---|---|---|---|---|---|
| Extent of dilution of | ASGPR positive samples | | | | | ASGPR negative samples | | | | |
| secondary antibody | 1:30 | 1:50 | 1:100 | 1:200 | 1:400 | 1:30 | 1:50 | 1:100 | 1:200 | 1:400 |
| 1:1000 | 1.587 | 1.437 | 1.134 | 0.878 | 0.456 | 0.297 | 0.263 | 0.197 | 0.185 | 0.155 |
| 1:2000 | 1.347 | 1.104 | 0.996 | 0.746 | 0.376 | 0.261 | 0.186 | 0.172 | 0.153 | 0.135 |
| 1:4000 | 0.896 | 0.702 | 0.615 | 0.518 | 0.315 | 0.243 | 0.174 | 0.159 | 0.147 | 0.117 |
| 1:6000 | 0.587 | 0.474 | 0.397 | 0.328 | 0.267 | 0.185 | 0.167 | 0.146 | 0.135 | 0.105 |

TABLE 9
P/N value of checkerboard titration

| | P/N | | | | |
|---|---|---|---|---|---|
| Extent of dilution | Extent of dilution of primary antibody | | | | |
| of secondary antibody | 1:30 | 1:50 | 1:100 | 1:200 | 1:400 |
| 1:1000 | 5.34 | 5.46 | 5.76 | 4.75 | 2.94 |
| 1:2000 | 5.16 | 5.94 | 5.79 | 4.88 | 2.79 |
| 1:4000 | 3.69 | 4.03 | 3.87 | 3.52 | 2.69 |
| 1:6000 | 3.17 | 2.84 | 2.72 | 2.43 | 2.54 |

It can be seen from the results that when the extent of dilution of the primary antibody is 1:50 and the extent of dilution of the secondary antibody is 1:2000, the P/N value reaches the maximum value.

### Example 11 Selection of TMB Development Time

The indirect ELISA test was performed under the above optimized optimal conditions. The final TMB development time was respectively set as 5 min, 10 min, 15 min, and 20 min, and then the development was stopped with 2 M H₂SO₄, the OD₄₅₀ value was determined with an ELISA, and finally the TMB development time was determined according to the P/N value.

TABLE 10
Detection results of different development time

| | | Samples | | | | |
|---|---|---|---|---|---|---|
| Development | | | | Negative | P/N | |
| time | | ASGPR1 | ASGPR2 | control | P1/N | P2/N |
| 5 | min | 1.125 | 0.658 | 0.136 | 8.27 | 4.83 |
| 10 | min | 1.478 | 0.945 | 0.174 | 8.52 | 5.45 |
| 15 | min | 1.787 | 1.220 | 0.202 | 8.87 | 6.05 |
| 20 | min | 2.185 | 1.471 | 0.256 | 8.54 | 5.74 |
| 25 | min | 2.339 | 1.603 | 0.289 | 8.09 | 5.55 |

When the development time is 15 min, the P/N value reaches the highest point. As the development time increases, the P/N value will decrease slightly.

### Example 12 Drawing of Standard Curve by ELISA Method

(1) Coating: GSA was diluted with a 0.05 M CBS buffered solution to 20 μg/mL to coat a 96-well ELISA plate with 100 μL per well at a temperature of 4° C. for 24 h, then washing was performed with a washing buffer solution (PBS containing 0.1% tween-20) three times, 3 min each time, and patting dry was performed.

(2) Blocking: 1% skim milk was added to the coated ELISA well plate with 300 μL per well, and the plate as sealed with a microplate sealer, and blocked at a temperature of 37° C. for 2 h; a blocking buffer was blocked, washing was performed with a washing buffer solution three times, 3 min each time, and patting dry was performed;

(3) Sample adding: an ASGPR negative control well and ASGPR standard product wells were respectively set, and the standard products were respectively diluted with a standard diluent to seven concentrations, i.e., 5 μg/L, 10 μg/L, 20 μg/L, 40 μg/L, 60 μg/L, 80 μg/L, and 100 μg/L. The negative control and ASGPR standard products were sequentially added to the 96-well ELISA plate, replicate wells were made, and the sample adding volume for each well was 100 μL. After adding the sample, the plate was sealed with a microplate sealer, and incubation was performed at a temperature of 37° C. for 2 h.

(4) Primary antibody incubation: a mouse-derived primary antibody of ASGPR1/2 was incubated by diluting with a blocking buffer according to a ratio of 1:50, with 100 μL per well, at a temperature of 37° C. for 2 h, then washing was performed with a washing buffer solution four times, 3 min each time, and patting dry was performed;

(5) Secondary antibody incubation: an HRP-labeled goat anti-mouse enzyme-labeled secondary antibody (Kangwei Century Biotechnology Co., Ltd., CW0102) was incubated by diluting with a blocking buffer according to a ratio of 1:2000, with 100 μL per well, at a temperature of 37° C. for 1 h, then washing was performed with a washing buffer solution four times, 3 min each time, and patting dry was performed;

(6) TMB development: the development was performed with a TMB development solution (Biyuntian Biotechnology Co., Ltd., P0209), with 200 μL per well, incubation was performed for 15 min in the dark, and then immediately the development was stopped with 2 M H₂SO₄, with 50 μL per well; after the development was stopped, the absorbance of each well at 450 nm was measured with an ELISA as soon as possible.

(7) Standard curve drawing: the OD₄₅₀ values of different concentrations of ASGPR standard products were read and a standard curve was drawn.

### Example 13 Determination of Minimum Limit of Detection

According to the method of Example 12, the ASGPR negative control sample was repeatedly determined on the same coated ELISA plate for 20 wells, and the determination was repeated for five batches. The average value M and standard deviation SD of the OD₄₅₀ values of the negative control sample were calculated, the value of M±3SD was calculated, and substituted into the equation of the standard curve to obtain the minimum limit of detection of the ELISA method. The result shows that the minimum limit of detection is 4 μg/mL.

TABLE 11
Detection results of repeated 5 batches

| | First | Second | Third | Fourth | Fifth |
|---|---|---|---|---|---|
| | time | time | time | time | time |
| Average value | 0.151 | 0.162 | 0.169 | 0.155 | 0.153 |
| Standard deviation | 0.005 | 0.005 | 0.004 | 0.004 | 0.005 |
| Minimum limit of | 3.41 | 3.75 | 3.56 | 2.95 | 3.36 |
| detection (μg/L) | | | | | |

### Example 14 Inter-Batch and Intra-Batch Repeat Tests

By using GSA coated ELISA plates prepared in the same batch and different batches, the repeat tests were performed on three different concentrations of ASGPR positive samples, negative samples, and serum samples according to the method of Example 12, three times for each; after the OD value was determined, the average values of coefficients of variation of the inter-batch and intra-batch repeat tests were both less than 7% by calculating, so the method has good repeatability.

TABLE 12
Inter-batch repeated tests

| | | | | | | Inter-batch |
|---|---|---|---|---|---|---|
| | | | | | | coefficient |
| | Repeat times | | | | | of |
| | First | Second | Third | Average | Standard | variation |
| Samples | time | time | time | value | deviation | (%) |
| Negative | 0.184 | 0.169 | 0.164 | 0.173 | 0.009 | 5.28% |
| control | | | | | | |
| ASGPR1 | 0.229 | 0.251 | 0.225 | 0.240 | 0.014 | 5.64% |
| ASGPR2 | 0.833 | 0.906 | 0.801 | 0.853 | 0.041 | 4.76% |
| ASGPR3 | 1.444 | 1.459 | 1.369 | 1.410 | 0.076 | 5.40% |
| Serum | 0.766 | 0.722 | 0.752 | 0.755 | 0.019 | 2.56% |
| sample 1 | | | | | | |

TABLE 13
Intra-batch repeated tests

| | | | | | | Intra-batch |
|---|---|---|---|---|---|---|
| | | | | | | coefficient |
| | Repeat times | | | | | of |
| | First | Second | Third | Average | Standard | variation |
| Sample | time | time | time | value | deviation | (%) |
| Negative | 0.154 | 0.145 | 0.164 | 0.158 | 0.009 | 5.68% |
| control | | | | | | |
| ASGPR1 | 0.211 | 0.191 | 0.223 | 0.211 | 0.013 | 5.91% |
| ASGPR2 | 0.713 | 0.716 | 0.731 | 0.730 | 0.016 | 2.14% |
| ASGPR3 | 1.245 | 1.25 | 1.269 | 1.226 | 0.042 | 3.44% |
| Serum | 0.689 | 0.653 | 0.623 | 0.644 | 0.029 | 4.56% |
| sample 1 | | | | | | |

### Example 15 Linear Range

Three batches of coated ELISA plates were used to detect 5 different concentrations (5 μg/L, 10 μg/L, 20 μg/L, 40 μg/L, 70 μg/L, and 100 μg/L) of ASGPR standard products, the determined concentration and the theoretical concentration were linearly fitted according to the method of Example 12, and the detection was repeated three times. The linearly dependent coefficients r of fitted straight lines obtained by determining three times are all greater than 0.990, so the method satisfied the expected set linear range of 4 to 100 μg/mL.

### Example 16 Accuracy (Recovery Test)

Three serum samples were taken, and ASGPR standard products having high (20 μg/mL), medium (10 μg/mL) and low (5 μg/mL) concentrations were respectively added to the three serum samples, the sASGPR concentrations of the original serum sample and the samples added with ASGPR were detected according to the method of Example 12, each sample was detected three times repeatedly, and the recovery volume and recovery rate of ASGPR were calculated.

TABLE 14
Recovery test

| | ASGPR (μg/L) | | | |
|---|---|---|---|---|
| | Addition | Detection | Recovery | Recovery |
| Samples | volume | volume | volume | percentage (%) |
| Serum sample 1 | 0 | 78.86 | — | — |
| Serum sample 1 | 5 | 83.43 | 4.57 | 91.40% |
| Serum sample 1 | 10 | 87.89 | 9.03 | 90.30% |
| Serum sample 1 | 20 | 97.30 | 18.44 | 92.20% |
| Serum sample 2 | 0 | 85.22 | — | — |
| Serum sample 2 | 5 | 90.10 | 4.88 | 97.60% |
| Serum sample 2 | 10 | 94.46 | 9.24 | 92.40% |
| Serum sample 2 | 20 | 104.20 | 18.98 | 94.90% |
| Serum sample 3 | 0 | 108.87 | — | — |
| Serum sample 3 | 5 | 113.20 | 4.33 | 86.60% |
| Serum sample 3 | 10 | 117.66 | 8.79 | 87.90% |
| Serum sample 3 | 20 | 128.89 | 20.02 | 100.10% |
| Average recovery | | | | 92.60% |
| rate | | | | |

### Example 17 Detection of Clinical Serum Samples by ELISA Method

According to the method of Example 12, the ASGPR negative control sample, the ASGPR standard products and the samples to be detected (3 serum samples) were respectively detected, the standard products were respectively diluted with a standard diluent to seven concentrations, i.e., 5 μg/L, 10 μg/L, 20 μg/L, 40 μg/L, 60 μg/L, 80 μg/L, and 100 μg/L, and the serum samples were diluted two times with 1×PBS for later use. A standard curve was drawn and the ASGPR content of the corresponding sample was calculated according to the OD₄₅₀ value of the sample well.

TABLE 15
Detection of serum samples by ELISA

| Standard (μg/L) | OD₄₅₀ | Serum samples (μg/L) | OD₄₅₀ |
|---|---|---|---|
| 0 | 0.164 | 80 | 1.563 |
| 5 | 0.239 | 100 | 1.866 |
| 10 | 0.364 | Serum sample 1 | 0.793 |
| 20 | 0.494 | Serum sample 2 | 0.857 |
| 40 | 0.806 | Serum sample 3 | 0.684 |
| 60 | 1.188 | Serum sample 3 | 0.924 |

It can be seen from the above table that the OD values of the serum samples are within the test range of the standard curve, so the method can achieve the expected detection effect.

### Example 18 Detection of Clinical Serum Samples by ELISA Method

A total of 533 serum samples were detected by the indirect ELISA method of Example 12, including 489 serum samples of healthy people and 44 serum samples of liver injury patients. The detection results show that the content of sASGPR in the serum samples of healthy people is 85.94±59.69 μg/mL, and the content of sASGPR in the serum samples of liver injury patients is 20.41±10.59 μg/mL. By statistical analysis, there was a statistically significant difference in content of serum sASGPR between the healthy samples and liver injury samples (P<0.001).

Although the disclosure has been disclosed in the above as preferred embodiments, it is not intended to limit the disclosure. Those skilled in the art can make various changes and modifications without departing from the spirit and scope of the disclosure. The protection scope of the disclosure shall be defined by the claims.

What is claimed is:

1. A method for detecting a human soluble asialoglycoprotein receptor (ASGPR), comprising using an (enzyme-linked immunosorbent assay (ELISA method) to detect galactosylated human serum albumin (GSA) as a specific ligand of ASGPR.

2. The method according to claim 1, wherein the using the ELISA method to detect GSA comprises: coating an ELISA plate with GSA diluted with a CBS buffered solution at a temperature of 0 to 4° C. for 12 to 24 h, and then washing with a washing buffer; adding a blocking buffer to the coated ELISA plate to block at a temperature of 35 to 37° C. for 1.5 to 2.5 h; discarding the blocking buffer, and washing with a washing buffer; adding a sample to the ELISA plate, incubating at a temperature of 35 to 37° C. for 2 to 2.5 h, then washing with a washing buffer, and patting dry; adding an ASGPR1 primary antibody and incubating at a temperature of 35 to 37° C. for 2 to 2.5 h, and then washing with a washing buffer; adding an HRP-labeled goat anti-mouse enzyme-labeled secondary antibody, incubating at a temperature of 35 to 37° C. for 1 to 2 h, and washing with a washing buffer; developing with a TMB development solution, incubating for 10 to 20 min in the dark, and then immediately stopping the development with H₂SO₄; and determining absorbance at 445 to 455 nm.

3. The method according to claim 2, wherein a concentration of the CBS buffered solution is 0.03 to 0.08 M.

4. The method according to claim 2, wherein a coating concentration of the GSA is 15 to 25 μg/m L.

5. The method according to claim 2, wherein the enzyme-labeled secondary antibody is added after being diluted 2000 times.

6. The method according to claim 2, wherein a concentration of the CBS buffered solution is 0.03 to 0.08 M; a coating concentration of the GSA is 15 to 25 μg/mL; and the enzyme-labeled secondary antibody is added after being diluted 2000 times.

7. A kit for detecting a human soluble asialoglycoprotein receptor, comprising a GSA-coated ELISA plate, a blocking buffer, an ASGPR standard product, an ASGPR primary antibody, a goat anti-mouse enzyme-labeled secondary antibody, a stop buffer and a washing buffer.

8. The kit according to claim 7, wherein the washing buffer comprises a first washing buffer and a second washing buffer; the first washing buffer is a PBS buffered solution; and the second washing buffer is a PBST buffered solution.

9. The kit according to claim 7, wherein the blocking buffer is a mixture of skim milk and a PBST buffered solution; and the stop buffer is an H₂SO₄ solution.

10. The kit according to claim 7, wherein the washing buffer comprises a first washing buffer and a second washing buffer; the first washing buffer is a PBS buffered solution; the second washing buffer is a PBST buffered solution; the blocking buffer is a mixture of skim milk and the PBST buffered solution; and the stop buffer is an H₂SO₄ solution.
